(19) United States
(12) Patent Application Publication
(10) Pub. No.: US 2010/0081637 A1
(43) Pub. Date: Apr. 1, 2010

(54) Eczema treatment with vitamin D and analogs thereof method, composition and cream

(75) Inventor: Marilyn Strube, Portland, OR (US)

Correspondence Address: ATER WYNNE LLP, 1331 NW Lovejoy St. Suite 900, PORTLAND, OR, 97209-2785, US

(73) Assignee: Innovia Skincare Corp.

(21) Appl. No.: 12/587,070

(22) Filed: Sep. 30, 2009

Related U.S. Application Data
(60) Provisional application No. 61/194,963, filed on Oct. 1, 2008.

(51) Int. Cl. A61K 31/59; A61P 17/00

(52) U.S. Cl. 514/167

(57) ABSTRACT

An eczema treatment method comprises application of a therapeutically effective composition in the form of an aqueous topical cream to an affected area of an exterior skin region of a patient, the aqueous topical cream comprising water, a water-soluble organic liquid, a surface active agent, and vitamin D₃ compound or another claimed analog thereof, the vitamin D₃ compound or the analog constituting at least approximately 10 international units per gram (IU/gram), and, most preferably at least approximately 400 IU/gram. An eczema treatment composition comprises a therapeutically effective formulation comprising at least approximately 50%-90% water, between approximately 1-5% PEG-40 stearate, between approximately 1-5% steareth-2, and cholecalciferal at a concentration of at least approximately 50 international units per gram (IU/g) of composition, and, most preferably at least approximately 400 IU/g. A topical vitamin D cream for therapeutic treatment of eczema includes vitamin D or an analog thereof chosen from the group consisting of cholecalciferol, alfacalcidol, calcifedol, and ergocalciferol; water; a water-soluble organic liquid; and a surface active agent, the vitamin D or analog being in a concentration of at least approximately 50 international units per gram (IU/g) of cream. Remarkably, relief from eczema conditions occurs immediately or in less than 3 days.

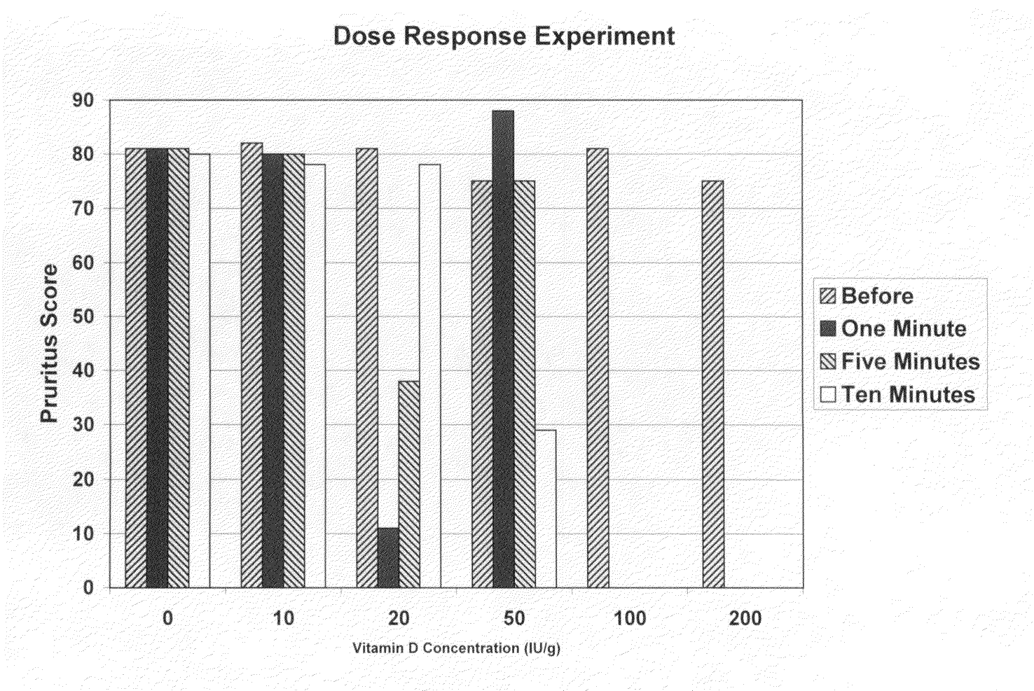

### RELATED APPLICATIONS

This application claims benefit of priority to U.S. Provisional Patent Application Ser. No. 61/194,963 filed Oct. 1, 2008, the contents of which are incorporated herein in their entirety by this reference.

### FIELD OF THE INVENTION

This invention relates generally to the field of the topical treatment of the eczema using vitamin D and analogs thereof.

### BACKGROUND OF THE INVENTION

Eczema is the term used to describe a number of skin conditions including contact dermatitis, atopic dermatitis and seborrhoeic dermatitis. These conditions are characterized by pruritus (itchiness), erythema (redness), excoriation (scratches, scabs), induration (swelling) and papulation (bumpiness) of the skin. Causes can range from allergic reactions to external substances, as in contact dermatitis, to internal causes as in atopic dermatitis or a yeast infection as in seborrhoeic dermatitis. Contact dermatitis occurs after exposure to substances such as urushiol oil from the poison ivy plant. Symptoms can include red, extremely itchy rashes, fluid filled blisters and swelling. Treatment for contact dermatitis involves removing the causative agent, then treating the remaining symptoms. Drying agents, such as calamine lotion are used to dry fluid filled blisters. Therapies include topical anti-inflammatory corticosteroids and numbing agents to help relieve swelling and itch. Seborrhoeic dermatitis can be treated with dandruff medications, anti-fungals and corticosteroids.

Although the exact cause of atopic dermatitis is unknown, it is thought to be the result of a disorder of the immune system and appears to have a genetic component, often occurring in great frequency in certain families and ethnic groups. It often presents during infancy and sometimes disappears by adulthood. In some cases, it presents for the first time in adulthood. It can cause considerable distress from constant itchiness, dry skin, unsightly rashes and, in the worst cases, can cause debilitation and require hospitalization. There is no known cure for atopic dermatitis. Treatments consist of moisturizing lotions, occlusive dressing for extremely dry patches, topical anti-inflammatory corticosteroids and numbing agents. OTC strength preparations of these therapies usually have a minimal effect. Prescription strength corticosteroids are the most commonly prescribed therapies but can have serious detrimental side effects such as thinning of the skin, stretch marks or eye damage often necessitating discontinuation of the treatment. Numbing agents and antihistamines provide minimal relief in the majority of moderate to severe clinical cases. Soothing emollients, phenol, menthol and camphor can provide some short term relief for mild pruritus. UVB light therapy is commonly used to treat severe cases of eczema with a high rate of effectiveness, although undesirable side effects can occur such as an increased risk of developing skin cancer as well as undesirable phototoxic reactions (see for example, Marks, J Dermatol Treat 1:233-234, 1989).

It was discovered that a topical oil-in-water emulsion formulation containing a rich concentration (400 IU) of vitamin D3, (cholecalciferol) sometimes referred herein simply as vitamin D, quickly and completely relieved severe pruritus associated with chickenpox with relief lasting for hours. Subsequently a similar formulation provided significant or total relief of pruritus in several additional cases of chickenpox, followed by 51 out of 77 cases (66%) associated with a variety of additional pruritic conditions including atopic eczema, contact dermatitis, diabetic neuropathy, urticaria and psoriasis (results not published).

Our invention is described in U.S. Provisional application No. 60/005,030, filed on 10 Oct. 1995, which matured into U.S. Pat. No. 5,789,399 entitled TREATMENT OF PRURITUS WITH VITAMIN D AND ANALOGS THEREOF, the disclosures of which are hereby incorporated herein in their entirety by this reference.

The previous invention, cited in the US patent above, describes the utility of vitamin D₃ in the treatment of pruritus when administered in a topical formulation. Since then, two similar cholecalciferol (400 IU/g) formulations were recorded to have a significant healing effect in five cases of eczema with respect to one or more of the following symptoms: erythema, excoriation, induration and papulation (see Examples 1-5 below). However, in one case of severe atopic dermatitis, significant relief of pruritus with no healing effect was observed (see Example 6 below). While it can be expected that a reduction in scratching can lead to a decrease in excoriation, the overall fast and significant reduction in erythema, induration and papulation was not noted in several cases of chickenpox and at least not reported in some cases of eczema, and therefore was wholly unexpected. As cited in the previous patent, the effectiveness of UVB light therapy in the treatment of pruritus had been noted to be incidentally related to the production of vitamin D in the skin (Blachley et al., Am J Kidney Dis 5:237-241, 1985). The same wavelengths of between 290 and 310 nm used in UVB treatment were also the most effective wavelength for the conversion of provitamin D₃ (7-dehydrocholesterol) to vitamin D₃ (cholecalciferol). Nevertheless no direct link between vitamin D₃ production in the skin and the effectiveness of UVB light treatment have been proposed other than in the prior patent and, in addition, the mechanism of additional therapeutic benefits of UVB light remained unknown.

Thus, although vitamin D₃ had been incidentally related to some treatment modalities, i.e., UV light therapy, it had not previously been known that vitamin D₃ can be effectively used directly to treat any conditions or symptoms. The above-cited and incorporated US patent changed all that. The patent established that successful treatment of pruritis with vitamin D and its analogs in a topical formulation is possible. The patent nevertheless did not demonstrate any evidence, nor were the authors of the patent aware, that the use of this treatment would have a positive therapeutic effect on the underlying physiological condition that gives rise to erythema, excoriation, induration, papulation or other visible symptoms of eczema.

There are conflicting reports on the benefits of the use of “active” metabolites of vitamin D for the treatment of eczema. Renowned vitamin D researcher and physician, Dr. Michael F. Holick, gave an interview in 1997 excerpted from Nutrition Action Healthletter: Vitamin D Deficiency: the silent epidemic. Dr. Holick was asked if vitamin D creams treat psoriasis, answered “Yes, but you need a prescription medication for an active vitamin D analog, which is called Dovonex. You can't do it on your own by taking excessive amounts of the regular vitamin D or using a cream that contains it. We've also tried activated vitamin D on eczema, and it's not effective.” (Emphasis added.)

Contrary to Dr. Holick's comment, Kiyofumi Egawa reported that five patients treated with calcipotriol 50 ug/g and maxacalitriol 25 ug/g had an improvement of lesions from atopic dermatitis 2 to 8 weeks after treatment. There are distinctions between the claimed eczema treatment and the treatments described in the Egawa study. First, the Egawa study utilizes so called “active” double hydroxylated forms of vitamin D, whereas, the claims herein include only non or single hydroxylated forms. Second, the Egawa study utilized an ointment which typically implies an oil based formulation such as petroleum jelly or zinc oxide and the like. The previously cited patent provides teaching that vitamin D becomes bioavailable many times more efficiently in an oil-in-water emulsion cream. Finally, we report a significantly faster healing benefit from our formulations in treating eczema than the Egawa formulations, with relief occurring between 12 hours and a few days compared to 2 weeks and up. Calcipotriol and its synthetic derivatives have not been observed to have a direct, fast anti-pruritic benefit as do the vitamin D analogs claimed herein. The makers of Dovonex®, a psoriasis product containing calcipotriene, a synthetic derivative of calcitriol, lists pruritus as a potential side-effect and claim no anti-pruritic effect. As far as we are aware, there is no evidence that any double hydroxylated form of vitamin D has a significant, fast acting benefit in relieving pruritus or any other symptom of eczema. What we report here is a fast acting effect of the non and single hydroxylated forms of vitamin D3 on pruritus and other symptoms of eczema.

The apparent differences in anti-eczema activity between the single and double hydroxlyated forms of vitamin D may be due to in part to functional metabolic differences. A study (Nemere I. Apparent Normuclear Regulation of Intestinal Phosphate Transport: Effects of 1,25-Dihydroxyvitamin D3, 24,25-Dihydroxyvitamin D3 and 25-Hydroxyvitamin D3. Endocrinology. 1996 Vol. 137, No. 6. Pp 2254-2261) demonstrated important differences in the effects of 25-hydroxyvitamin D3 compared to 1,25-dihydroxyvitamin D₃ and 24,25-dihydroxyvitamin D₃ on phosphate transport in cultured chick duodenal loops. Unfortunately, cholecalciferol was not utilized in these tests but is closest in structure and function to 25-hydrovitamin D₃. “The presence or absence of luminal Ca did not influence phosphate transport in duodena vascularly perfused with control medium, 65 pM 1,25-dihydroxyvitamin D₃ or 6.5 nM 24,25-dihydroxyvitamin D₃. In contrast, vascular perfusion with 100 mM 25-hydroxyvitamin D₃ stimulated phosphate transport with luminal Ca was present but not when it was absent.” Ibid. This suggests a Ca dependent phosphate transport mechanism mediated by 25-hydroxyvitamin D₃, but not 1,25-dihydroxyvitamin D₃ or 24,25-dihydroxyvitamin D₃. Additionally, a subnormal concentration (20 nM) 25-hydroxyvitamin D₃ was shown to have the curious effect of stimulating phosphate transport at much higher rates than did the normal concentration (100 nM). The author speculated that the enhanced phosphate transport seen at the subnormal concentration of 25-hydroxyvitamin D₃ may be a protective mechanism for organisms experiencing vitamin D deficiency. Our research and the Blachley study suggest that pruritus occurs under conditions of vitamin D₃ deficiency and the presence of Ca. Since 25-hydrovitamin D₃ is closer to vitamin D₃ in structure and function, the functional differences demonstrated in the Nemere study may account for apparent differences in effect on pruritus between the non or single hydroxylated and double hydroxylated forms of vitamin D3. The effect of vitamin D on other symptoms of eczema may or may not be related to phosphate metabolism. Additional functional differences between the vitamin D and its analogs may yet be discovered.

“Gentle Naturals Baby Eczema Cream” is formulated with petrolatum and dimethicone to form a barrier on the skin to prevent moisture loss. It contains aloe vera to help heal irritated skin, and calendula, jojoba oil, apricot oil, and soybean oil to soften and moisturize, plus vitamins A, D, and E to nurture skin and help it heal. But laboratory testing of this so-called “eczema cream” by independent laboratory BodyCote Testing Group of Portland, Oreg. reveals such low levels of vitamin D that the laboratory results listed “ERR” for Error in the % recovered of that substance, less than 6 IU/100 g. The laboratory's conclusion was that because the method reporting limit for vitamin D was 5 IU/g and the peak test results for vitamin D in the two tested cream samples were two orders of magnitude below, the test for vitamin D was “ND=None Detected.”

### SUMMARY OF THE INVENTION

In accordance with the prior patented invention, it was discovered that surprisingly, when vitamin D was administered in specific formulations to pruritic rashes, the vitamin D became therapeutically effective and the pruritus was rapidly and completely relieved. It was demonstrated that certain formulations are more suitable than others, presumably in that they provide more efficient absorption of vitamin D thus more effective at making it bioavailable. Additionally, the concentration of vitamin D was shown to be crucial, in that concentrations below certain levels were ineffective against pruritus, whereas concentrations at or above those levels were effective.

In accordance with the present invention, a surprising result has been discovered: aqueous topical dressings containing vitamin D and its analogs are effective in relieving visible symptoms of eczema, such as redness, scratches, scabs, swelling and bumpiness of the skin, in addition to the previously discovered anti-itch effect.

An oil-in-water emulsion formulation that is suitable for topical treatment was found to be essential for efficacy at lower vitamin D concentrations. This type of formulation is well known to be an effective carrier for fat soluble molecules like vitamin D. Thus, one formulation for vitamin D that was found to be effective in treating pruritus comprised a mixture of water, a water-insoluble organic liquid, a surface active agent, and cholecalciferol. Additional analogs claimed for this invention include the so called “inactive” forms of vitamin D3, cholecalciferol (vitamin D3), alfacalcidol (1-alpha-hydroxyvitamin D3), calcifediol (25-hydroxyvitamin D3) and ergocalciferol (vitamin D2). It is acknowledged herein that these analogs are inactive with respect to promotion of epidermal cell differentiation, inhibition of cell proliferation, inhibition of angiogenesis and modulation of cytokine production. However, that vitamin D3 is indeed active with respect to phosphate metabolism has been proven (Cross H., Peterlik M. Effects of vitamin D and insulin on phosphate transport in differentiating chick small intestine. Calcium and Phosphate Transport. 1981; 293-296), but not widely acknowledged. Whether phosphate activity or some other activity is the key to the anti-pruritic and healing effects of vitamin D is yet to be determined.

A second formulation that is also effective in treating pruritus due to eczema is based upon a non-aqueous carrier vehicle for vitamin D. This formulation comprises the water-insoluble organic petroleum jelly ointment, 20,840 IU/g vitamin D and provides fast relief of pruritus compared to petroleum jelly alone. Petroleum jelly can provide superior moisture retaining properties compared to a water based cream, making it a base of choice in some cases of extreme dry skin. A petroleum jelly formulation with a vitamin D concentration as high as 20,840 IU/g may prove useful as a remedy for pruritus and the outward symptoms of eczema. Among the several advantages achieved by the patented invention, therefore, may be noted the provision of a new treatment for eczema; the provision of a treatment modality that is safe being neither irritating nor toxic; the provision of a treatment that is inexpensive; and the provision of a treatment that has a low potential for sensitizing the treatment site by virtue of its being an endogenous or closely related to an endogenous substance.

### BRIEF DESCRIPTION OF THE DRAWING

The FIGURE is a graph illustrating the dose response experimental data in terms of pruritus scores on the vertical scale and a vitamin D concentration in international units per gram (IU/g) on the horizontal scale, with up to four different timing parameters represented by adjacent differentiated bars for each concentration. Pruritus relief is the fastest observed benefit after topical application of our claimed formulations. The graph below indicates an effective concentration as low as 50 IU/g. This is also an indication of bioavailability of vitamin D and therefore an indication of the lowest effective concentration for the treatment of additional symptoms of eczema.

### DETAILED DESCRIPTION OF THE PREFERRED EMBODIMENTS

The present invention is thus based upon the discovery that vitamin D can effectively treat some forms of eczema, such as atopic dermatitis, when administered topically in a therapeutically effective formulation. By a therapeutically effective formulation it is meant that the active substance is bioavailable, that it is able to exert a biological activity when administered in that formulation. Such formulations effectively exhibit the biological activity of vitamin D in diminishing or completely alleviating the underlying physiological conditions of, for example, eczema. The formulation is also pharmaceutically acceptable for topical application.

Vitamin D within the scope of this invention is intended to include vitamin D₃ or cholecalciferol as well as alfacalcidol, calcifedol and ergocalciferol.

Also included within the scope of this invention and within the meaning of the term vitamin D are analogs of vitamin D. Analogs of vitamin D are well known in the art and such compounds can have modifications in the side chain and/or modifications in the nuclear part of the secosteroid molecule. Vitamin D analogs can contain side chain modifications involving introduction of unsaturation; substituting hydrogens; forming carbocyclic structure; introducing a heteroatom link; inverting stereochemically; removing or adding alkyl groups and changing the number of links in the side chain. (see Calverly and Jones, in Antitumor Steroids blickenstagg, Ed., Academic Press, Inc., San Diego, 1992, 193-270 which is incorporated herein by reference). All of these compounds are thus included in the term vitamin D so long as they exhibit the biological activity of anti-eczema effectiveness in a bioavailable formulation. Such biologically active vitamin D compounds may not show the same degree of activity as vitamin D₃, but need only show at least some discernable anti-eczema effect.

It is believed that, at least with respect to the anti-pruritic mechanism and perhaps to the other healing effects of this invention, vitamin D is applied to the skin in a therapeutically effective formulation, then directly enters the patient's skin cells to beneficially decrease elevated cellular phosphate compounds to normal levels, although applicant does not intend to be limited to such a theory of operation or physiological modus operandus.

One embodiment of the present invention is an aqueous-based emulsion formulation comprising water, a water-insoluble organic liquid, a surface active agent and vitamin D. The surface active agent is biocompatible and suitable for application by topical formulation. The formulation also contains a biocompatible water-insoluble organic liquid such as an oil or lipid suitable for forming an oil-in-water or water-in-oil emulsion so that the formulation is a cream, ointment, paste or the like. By biocompatible it is meant that the substance produces no untoward biological effects such as local or generalized toxic effects or local irritation at the site of topical application. Suitable water-insoluble organic liquids are well known in the field of topically applied cosmetics and therapeutics and include but are not limited to mineral oil, corn oil or other vegetable oil, petroleum, lanolin, fish oil and the like.

The use of surface active agents in preparations that are topically applied is well known particularly in the field of cosmetics (for example, see Surfactants in Cosmetics, Rieger, M., Ed., Marcel Dekker, Inc., N.Y., 1985 which is incorporated by reference). Thus, a number of biocompatible surface active agents can be used in the present invention including but not limited to (1) anionic surfactants such as monovalent or polyvalent carboxylate salts, acyl lactylates, alkyl ether carboxylates, N-Acyl Sarcosinates, N-Acyl Glutamates, fatty acid-polypeptide condensates, sulfuric acid esters including alkyl sulfates and ethoxylated alkyl sulfates, ester-linked sulfonates, alpha olefin sulfonates, phosphated ethoxylated alcohols; (2) cationic surfactants such as monoalkyl quaternary ammonium salts, dialkyl quaternary ammonium compounds, amidoamines and aminimides; (3) amphoteric surfactants such as N-substituted alkyl amides, N-alkyl betaines, sulfobetaines, and N-alkyl beta-aminopropionates; and (4) nonionic surfactants such as (a) polyoxyethylene compounds including ethoxylated alcohols, ethoxylated esters and ethoxylated amines; (b) polyoxypropylene compounds such as propoxylated alcohols, ethoxylated/propoxylated block polymers and propoxylated esters; (c) alkanolamines; (d) amine oxides; and (e) fatty acid esters of polyhydric alcohols such as ethylene glycol esters, diethylene glycol esters, propylene glycol esters, glyceryl esters, polyglyceryl fatty acid esters, sorbitan esters, sucrose esters and glucose esters.

The selection of a particular surface active agent would depend upon a number of factors including but not limited to the particular causation agent for the dermatitis condition and whether topical lesions accompany the dermatitis condition, e.g. the eczema. The formulations of the present invention are useful in treating dermatitis conditions on the skin, mucous membranes or cornea. Selection of a suitable surface active agent can thus be based in part on the site being treated. For example, amphoteric or nonionic surface active agents known in the art are selected for uses in and around the eyes to avoid corneal irritation. One skilled in the art can readily select a surface active agent and formulation suitable for a particular site and dermatitis condition as well as for other factors.

In another embodiment of the present invention a water-insoluble formulation is provided comprising a biocompatible, water-insoluble organic liquid such as a biocompatible oil or lipid suitable as a carrier for the vitamin D. The carrier must be suitable in the sense of being compatible with the vitamin D and any other ingredients and not deleterious to the patient being treated. Suitable organic liquids include petroleum jelly, corn oil or other vegetable oil, mineral oil, lanolin, fish oil and the like.

The vitamin D compounds of the present invention can also be formulated into bioavailable suspensions or dispersions such as, for example, hydrosols and hydrogels in which a true emulsion is not formed. The formulation is a mixture of two or more substances forming a dispersed phase which is finely divided and uniformly distributed through a dispersion medium which is usually water. The dispersed phase can be a solid or an oil phase with stabilizers to form a stabilized colloidal system. Suspending or dispersing agents may also be added to the formulation and can include surface active agents capable of stabilizing the suspension or dispersion. The preparation of suspensions and ingredients used in the formation of suspensions are well known in the art and one skilled in the art can readily prepare such suspension.

In addition to the above ingredients, the formulations of this invention may also include one or more additional auxiliary ingredients such as diluents, buffers, or preservatives such as methyl hydroxybenzoate and/or anti-oxidants and the like. Such additional ingredients can also be pharmaceutically-acceptable excipients for modifying or maintaining the pH, osmolarity, viscosity, clarity, color, sterility, stability, rate of dissolution, or odor of the formulation. Similarly, the formulation may contain still other pharmaceutically-acceptable excipients for modifying or maintaining release or absorption or penetration of the site by the vitamin D compound. Moreover, other therapeutic substances can be incorporated into the formulation such as, for example, other anti-dermatitis agents, vitamins, anti-fungals, anti-inflammatory agents, antibiotics, sunscreens and the like.

The topical formulations described herein can be used in treating virtually any dermatitis site such as for example on the skin, mucous membranes or cornea. Such treatments include scalp treatment as in shampoos, conditioners and the like, rectal treatment, eye treatment, treatment of nasal membranes, treatment of the ear canal, etc.

The formulations in the present invention are intended for use in treating a wide variety of dermatitis conditions, e.g. eczema, in humans as well as in non-human mammals, birds, reptiles, amphibians and fish.

Preferred embodiments of the invention are illustrated in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the Examples 1-5 regarding the effective treatment of and relief from eczema.

### Example 1

Female, 11 years of age, diagnosed with atopic dermatitis at age of 3. Before treatment, the patient experienced moderate pruritus daily over 90% of her body and extensive erythema, excoriation and induration/papulation on her arms, legs and neck. Prior treatments consisted of “any and all over the counter creams” and Fluticasone 0.005%, a prescription hydrocortisone preparation. A vitamin D (400 IU/g) cream was applied. The patient reported that the cream not only relieved her itch but also provided substantial improvement to all symptoms after a treatment period of 2-3 days. The vitamin D cream was judged to work “much better” than all past treatments. Patient's mother's comment: “We are so happy that ______ can now wear shorts and sleeveless shirts. Before she was so scabbed and red, people would stare so she would cover it up with clothes.”

### Example 2

Female, 29 years of age, diagnosed with atopic dermatitis at age of 15. Before treatment, the patient suffered from pruritus, erythema and induration/papulation on her hands and eyelids. Prior treatment consisted of “Working Hands” a moisturizing cream. A vitamin D (400 IU/g) cream was applied. The patient reported that the cream provided substantial improvement to all symptoms after a treatment period of 1 day. The vitamin D cream was judged to work “much better” than past treatments. Patient's comment: “Really impressed and appreciated effectiveness!”

### Example 3

Female, 2½ years of age, diagnosed with atopic dermatitis at 1½ years of age. Before treatment, according to her mother, the patient experienced severe, constant pruritus over 95% of her body, erythema and excoriation over 60% of her body and induration/papulation over 40% of her body. Prior treatments consisted of:

Hydrocort Val 02%—did not help with the itch or redness
Zyrtec—made her sleepy but did not help with the itching
Hydroxyz Hcl 10 mg—made her sleepy but did not help with the itching
Plastibase Ointment—no effect
Elidel (pimecromlimus) cream 1%—no effect
Desonide 0.5%—gives itch relief and reduces redness
By use of the invented method, composition and cream, the patient showed substantial improvement of pruritus, erythema and excoriation and some improvement of induration/papulation.

Comparison of the vitamin D treatment with past treatments:

“Much better than Zyrtec, Hydrocort Val 02%, Hydroxyz Hcl 10 mg, Elidel (pimecromlimus) cream 1% and Plastibase ointment. Comparable to the Desonide 0.05% . . . although relief of the itching is FASTER with the vitamin D cream.”

Comments: “We have spent hundreds of dollars trying to help our small daughter with this cruel condition. We purchased just about every over-the-counter lotion and cream that claim to treat eczema, with no success. ______ has been treated by an allergist and a pediatric dermatologist. None of the prescribed treatments truly gave her relief except the stronger steroid cream (Desonide 0.05%). We don't want to use a steroid cream on our child indefinitely. (Frankly, we don't know any parent who WANTS to use a steroid cream.) However, you are often made to feel there are no other alternatives in the medical arena. I was given a sample of the vitamin D cream by her Pediatrician and was thrilled with the results! I noticed a change in the texture and redness of her skin by the next morning. We apply the cream after bathing, before bedtime, and during the night, when needed for itching. I stopped using the steroid cream completely and substitute with the vitamin D cream. I've found it to be comparable, if not better! It will be wonderful when there is a cure for atopic dermatitis, but until then we prefer a natural and effective treatment for our daughter's skin. Thank you!”

### Example 4

Seven year old male with atopic dermatitis from age two. A vitamin D (400 IU/g) cream was applied to the affected areas of the subject's skin. The vitamin D cream relieved the itch immediately and inflammation was gone overnight after the first application. Comments: “He has dug so hard into his skin, that he got scarred and raw. Sometimes he scratched at night and we had to worry about infection. I have been looking for something to relieve his itch for five years, but nothing helped. The Cream worked totally better than anything. We also noticed that the inflammation went away overnight with the first application. I'm a happy, less stressed mother! Thank you!”

### Example 5

Adult male. Reported that he had eczema for 6 months with “nightly” episodes of pruritus. He reported that he was using Fluocinonide 0.05% and reported it to be “fairly effective”. A vitamin D (400 IU/g) cream was applied. He reported that substitution with the vitamin D cream provided “total relief” of itch that lasted “all day” and stated that it was “much better” than his past treatments. He commented that it “seems to promote healing as good as prescription medicines”.

### Example 6

Female, 33 years old with a long history of very severe atopic dermatitis. She reported many severe episodes of the disease that required hospitalization. Use of the vitamin D (400 IU/g) cream relieved itch in seconds with relief lasting for 2-3 hours. She reported that there was no effect on the accompanying rash. She wrote “Nothing, nothing else stops the itch, so this certainly is sensational!!!”

In summary, the invented treatment method and composition provide an active, direct structure/function therapeutic effect (believed to be analogous to phosphate “clearing” like that provided by UVB light therapy). It does so without the attendant melanoma risks of exposure to UV light. It does so in a rich concentration (preferably at least approximately 50 international units per gram (IU/g), more preferably at least approximately 100 IU/g, even more preferably at least approximately 200 IU/g, and most preferably approximately 400 IU/g) of vitamin D in an oil-water emulsion cream for topical application to a patient suffering from eczema. The oil-water emulsion to which such concentrations of vitamin D are added to produce an effective treatment composition or cream may include, for example, approximately 50-90% water by weight or volume, approximately 1-5% PEG-stearate by weight or volume, and approximately 1-5% steareth-2 by weight or volume, or suitably alternative components in suitably alternative weight or volume ranges.

The mechanism for the treatment of eczema may be different from that for the treatment of pruritis. It is believed that the invented composition provides a sufficient quantity of vitamin D to enter the patient's skin cells and to become bio-available beneath the stratum corneum. It is unprecedented to use vitamin D as more than a mere skin protectant, which is how the US Food and Drug Administration (FDA) classifies it. Thus it is surprising that vitamin D in a topical application is therapeutic and effective in treating eczema. It is believed that the penetrative vitamin D metabolizes phosphate in the skin cells and restores phosphate to acceptable levels the patient's previously pathological levels. Nevertheless, applicant does not intend to be limited to a particular effective-treatment modality or theory.

Such effective eczema treatment thus involves applying such a composition to the outer surface of the patient's skin (and optionally waiting and/or observing) for up to 24 hours to 2 or 3 days or more (depending in part on the vitamin D concentration), until the topical treatment has achieved a level of therapeutic effectiveness in treating the externally observable symptoms of the underlying eczema condition. Additional applications may be applied as needed.

It is believed from the Examples above that relief from eczema can be immediate, and usually does not require such a waiting period. Thus, it is concluded that the invented vitamin D (400 IU/g) cream treatment is fast-acting.

As various changes could be made in the above methods, compositions and creams without departing from the scope of the invention, it is intended that all matter contained in the above description and shown in the accompanying drawings shall be interpreted as illustrative and not in a limiting sense.

It will be understood that the present invention is not limited to the method or detail of application or composition or concentration or cream or wait period. Indeed, any suitable variation of method of use, application, composition, cream, or concentration is contemplated as an alternative embodiment, and thus is within the spirit and scope, of the invention.

Accordingly, while the present invention has been shown and described with reference to the foregoing eczema treatment methods, composition and cream of the invented apparatus, it will be apparent to those skilled in the art that other changes in form and detail may be made therein without departing from the spirit and scope of the invention as defined in the appended claims.

I claim:

1. An eczema treatment method comprising:
applying a therapeutically effective composition in the form of an aqueous topical cream to an affected area of an exterior skin region of a patient, the aqueous topical cream comprising water, a water-soluble organic liquid, a surface active agent, and vitamin D₃ compound or another claimed analog thereof, the vitamin D₃ compound or the analog constituting at least approximately 50 international units per gram (IU/gram).

2. The method of claim 1, wherein the vitamin D₃ compound or its analog constituting at least approximately 100 IU/gram.

3. The method of claim 2, wherein the vitamin D₃ compound or its analog constituting at least approximately 400 IU/gram.

4. The method of claim 3 further comprising:
waiting after the application a period of time effective to treat the patient's eczema.

5. The method of claim 4, wherein the waiting is for less than approximately 3 days.

6. The method of claim 4, wherein the waiting is for less than approximately 2 days.

7. The method of claim 4, wherein the waiting is for less than approximately 24 hours.

8. The method of claim 1, wherein the vitamin D₃ compound or its analog is an activated vitamin D₃ compound of cholecalciferol.

9. The method of claim 1, wherein the therapeutically effective formulation comprises at least approximately 50% water, between approximately 1-5% PEG-40 stearate, between approximately 1-5% steareth-2, and cholecalciferol at a concentration of approximately 400 international units per gram (IU/g).

10. An eczema treatment composition comprising:
a therapeutically effective formulation comprising at least approximately 50% water, between approximately 1-5% PEG-40 stearate, between approximately 1-5% steareth-2, and cholecalciferol at a concentration of at least approximately 50 international units per gram (IU/g) of the composition.

11. The composition of claim 10, wherein the concentration is of at least approximately 100 IU/g of the composition.

12. The composition of claim 11, wherein the concentration is of at least approximately 200 IU/g of the composition.

13. The composition of claim 12, wherein the concentration is of at least approximately 400 IU/g of the composition.

14. An eczema treatment method comprising:
applying a therapeutically effective composition in the form of an aqueous topical cream to an eczema-affected area of an exterior skin region of a patient, the aqueous topical cream comprising water, a water soluble organic liquid, a surface active agent, and vitamin D₃ compound or an analog thereof, the vitamin D₃ compound or the analog thereof constituting at least approximately 50 international units per gram (IU/g) of cholecalciferol.

15. The method of claim 14, wherein the vitamin D₃ compound or its analog constitutes at least approximately 100 IU/g of cholecalciferol.

16. The method of claim 15, wherein the vitamin D₃ compound or its analog constitutes at least approximately 400 IU/g of cholecalciferol.

17. The method of claim 16 further comprising:
waiting after the application a period of time effective to treat the patient's eczema.

18. The method of claim 17, wherein the waiting is for less than approximately 3 days.

19. The method of claim 17, wherein the waiting is for less than approximately 2 days.

20. The method of claim 17, wherein the waiting is for less than approximately 24 hours.

21. A topical vitamin D cream for therapeutic treatment of eczema, the cream comprising:
vitamin D or an analog thereof chosen from the group consisting of cholecalciferol, alfacalcidol, calcifedol, and ergocalciferol;
water;
a water-soluble organic liquid; and
a surface active agent,
the vitamin D or analog being in a concentration of at least approximately 50 international units per gram (IU/g) of the cream.

22. The cream of claim 21, wherein the concentration is of at least approximately 100 IU/g.

23. The cream of claim 22, wherein the concentration is of at least approximately 200 IU/g.

24. The cream of claim 23, wherein the concentration is of at least approximately 400 IU/g.
